(19) United States
(12) Patent Application Publication
(10) Pub. No.: US 2019/0151320 A1
(43) Pub. Date: May 23, 2019

(54) APPLICATION OF MAPK SIGNALING PATHWAY INHIBITOR IN MANUFACTURE OF DRUGS FOR DELAYING DEGENERATION OF DOPAMINERGIC NEURONS

(71) Applicant: FUZHOU UNIVERSITY, Fuzhou, Fujian (CN)

(72) Inventor: Yufeng YANG, Fuzhou, Fujian (CN)

(21) Appl. No.: 16/091,438

(22) PCT Filed: Oct. 14, 2016
(86) PCT No.: PCT/CN2016/102154

§ 371 Date: Oct. 4, 2018

(30) Foreign Application Priority Data
Apr. 6, 2016 (CN) 201610208591.0
Apr. 6, 2016 (CN) 201610208614.8

(51) Int. Cl. A61K 31/519; A61P 25/16; A61K 31/277; A61K 31/166

(52) U.S. Cl. CPC A61K 31/519; A61K 31/166; A61K 31/277; A61P 25/16

(57) ABSTRACT

The present invention relates to the use of an MAPK signaling pathway inhibitor in the manufacture of a medicament for delaying the degeneration of dopaminergic neurons, in particular to the use in the manufacture of a medicament for treating PD. The MAPK signaling pathway inhibitor of the present invention can effectively delay the degenerative death of dopaminergic neurons and thus can fundamentally achieve the effect of treating PD. According to the present invention, the MAPK signaling pathway inhibitor is prepared into an oral dosage form and thus it does not need to be injected. The therapeutic effect on a patient can be achieved only by oral administration. According to the present invention, the MAPK signaling pathway inhibitor has a better therapeutic effect on PD caused by various causes and is of great significance for the treatment and cure of PD.

### CROSS REFERENCE

The present application claims the priorities of Chinese patent application 201610208614.8 submitted on Apr. 6, 2016 and Chinese patent applications 201610208591.0 submitted on Apr. 6, 2016, and all the contents of these two applications are hereby incorporated into the present application for reference.

### TECHNICAL FIELD

The present invention belongs to the technical field of medicine and specifically the present invention relates to the application of an MAPK signaling pathway inhibitor in the manufacture of a medicament for delaying the degeneration of dopaminergic neurons, in particular to the application in the manufacture of a medicament for treating Parkinson's Disease.

### BACKGROUND ART

Dopaminergic (dopamine, DA) neurons are closely related to Parkinson's Disease (PD). People first recognized PD (also known as “tremor palsy”) and then in the continuous exploration of its pathogenesis, began to understand the dopaminergic neurons and established their central role in the pathogenesis of PD. PD is a common degenerative disease of the central nervous system, which mainly affects middle-aged and elderly people. The majority of the patients come down with this disease after 50 years of age, but in recent years, younger people have been found to suffer from this disease. The main symptoms of the patients with PD are slow movements, tremors that cannot be controlled by the hands, feet or other parts of the body, body stiffness and balance disorders, leading to a failure to take care of themselves. According to statistics, the prevalence of PD in populations over the age of 60 in China is about 1.7%, there are more than 1 million patients in total, and there is an increase of at least 100,000 new patients each year. The degeneration and death of dopaminergic neurons in the substantia nigra of the midbrain and the significant reduction in the DA content of the striatum leading to incapability to meet the normal needs of the human bodies are one of the main causes of PD.

At present, PD is mainly treated by drugs or drugs assisted by surgeries. There are principally three commonly-used drugs: the first is a dopamine formulation such as Levodopa, which can provide a direct supplement of dopamine insufficiently secreted in the brain; the second is a drug that inhibits the degradation of dopamine, which can reduce the degradation of dopamine and relatively increase the dopamine content in the brain; the third is a dopamine receptor agonist that can enhance the absorption and utilization of dopamine in the brain. However, none of them can delay the degeneration of dopaminergic neurons and thus they cannot fundamentally achieve a reversing therapeutic effect.

Therefore, there is still a need for drugs that can delay the degeneration of dopaminergic neurons and fundamentally treat PD.

### SUMMARY OF THE INVENTION

It is an object of the present invention to provide a medicament capable of delaying the degeneration of dopaminergic neurons, particularly a medicament capable of treating PD, which is achieved by the Applicant in discovering a new use of the MAPK signaling pathway inhibitor by inventive effort.

Therefore, in one embodiment of the present invention, there is provided a method of delaying degeneration of dopaminergic neurons in 4 disease animal models in need thereof, the method comprising administering to the disease models a therapeutically effective amount of an MAPK signaling pathway inhibitor.

In another embodiment of the present invention, there is provided a method of treating PD in 4 disease animal models need thereof, the method comprising administering to the disease models a therapeutically effective amount of an MAPK signaling pathway inhibitor.

In still another embodiment of the present invention, there is provided a use of an MAPK signaling pathway inhibitor in the manufacture of a medicament for delaying degeneration of dopaminergic neurons.

In another embodiment of the present invention, there is provided a use of an MAPK signaling pathway inhibitor in the manufacture of a medicament for treating PD animal models.

According to one embodiment of the present invention, an MAPK signaling pathway inhibitor is provided for delaying the degeneration of dopaminergic neurons.

According to another embodiment of the present invention, an MAPK signaling pathway inhibitor is provided for treating PD animal models.

According to one aspect of the above embodiments of the present invention, the MAPK signaling pathway inhibitor comprises a MEK inhibitor and an ERK antagonist.

According to a specific embodiment of the present invention, the MAPK signaling pathway inhibitor is selected from U0126, PD0325901, trametinib or a combination thereof.

According to a particularly preferred embodiment of the present invention, the MAPK signaling pathway inhibitor is trametinib.

A significant advantage of the present invention is that the present invention provides a novel use of an MAPK signaling pathway inhibitor and specifically a use of an MAPK signaling pathway inhibitor for the manufacture of a medicament that delays the degeneration of dopaminergic neurons. The resulting product can effectively delay the degenerative death of dopaminergic neurons and fundamentally achieve the effect of treating PD animal models?. Moreover, the medicament does not need to be injected and the therapeutic effect can be achieved only by oral administration; the medicament has a better therapeutic effect on PD caused by various causes and is of great significance for the treatment and cure of PD.

### BRIEF DESCRIPTION OF THE DRAWINGS

FIG. 1 is a schematic diagram of the morphology of dopaminergic neurons in the local brain regions of the fruit flies treated with U0126.

FIG. 2 is a statistical diagram of the quantity of dopaminergic neurons in the local brain regions of the fruit flies treated with U0126. NS indicates no statistically significant difference; ** indicates a statistically significant difference according to student t-test, p<0.05.

FIG. 3 is a schematic diagram of the morphology of dopaminergic neurons in the local brain regions of the fruit flies treated with PD0325901.

FIG. 4 is a statistical diagram of the quantity of dopaminergic neurons in the local brain regions of the fruit flies treated with PD0325901. NS indicates no statistically significant difference; ** indicates a statistically significant difference according to student t-test, p<0.05.

FIG. 5 is a schematic diagram of the morphology of dopaminergic neurons in the local brain regions of the fruit flies treated with trametinib.

FIG. 6 is a statistical diagram of the quantity of dopaminergic neurons in the local brain regions of the fruit flies treated with trametinib. NS indicates no statistically significant difference; ** indicates a statistically significant difference according to student t-test, p<0.05.

### SPECIFIC EMBODIMENTS

In order to make the contents of the present invention easier to understand, the technical solutions described in the present invention are further described below in conjunction with specific embodiments, but the present invention is not only limited thereto.

### EXAMPLES

### Example 1: Effect of U0126

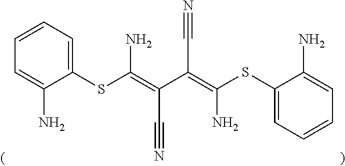 [SMILES: N#CC(=C(/N)Sc1ccccc1N)/C(C#N)=C(\N)Sc1ccccc1N]

### On Dopaminergic Neurons in Local Brain Regions

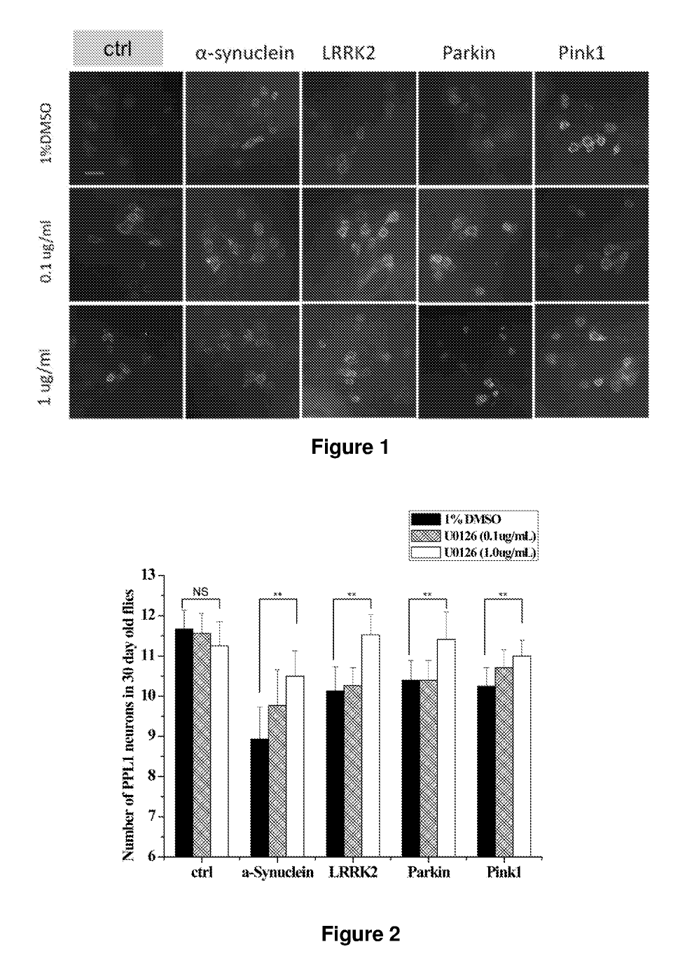

450 fruit flies of Drosophila melanogaster (purchased from Bloomington Drosophila Stock Center, USA) were divided into 4 PD disease model groups (as for “model groups”, each group has the same genotype and different groups have different genotypes; see the following) and 1 normal control group (Ctrl, non-PD type). Animal models of PD caused by alpha-synuclein transgene overexpression, Lrrk2 transgene overexpression, Parkin gene mutation and Pink1 gene mutation were established in the four model groups. Each group includes 90 fruit flies and each group was further equally divided into three groups, drug-feeding groups and non-drug-feeding control group, for testing. Starting from imago at the age of 5 days, the drug-feeding group was continuously fed with 0.1 μg/mL, 1 μg/mL U0126 (U0126 is dissolved in 1% DMSO) for 30 days and the non-drug-feeding control group was fed with 1% DMSO as a solvent control. After 30 days of drug or control solvent feeding, the brains of fruit flies were dissected and the dopamine neurons were labeled by the whole-tissue immunostaining of the brains of the fruit flies with an anti-dopamine hydroxylase antibody. The morphology and quantity of the dopamine neurons in the PPL regions in the brains of the fruit flies were observed microscopically to determine the effect of U0126 on the dopaminergic neurons in the local brain regions. The results thereof were shown in FIGS. 1 and 2.

FIG. 1 is a schematic diagram of the morphology of dopaminergic neurons in the local brain region after treatment with U0126, wherein the fluorescent part is the cell body profile of the dopaminergic neurons, through which the state and quantity of the dopaminergic neurons can be observed.

FIG. 2 is a statistical diagram of the quantity of the dopaminergic neurons in the PPL brain region after treatment with U0126.

As can be seen from FIGS. 1 and 2, 1 μg/mL U0126 can effectively reverse the degenerative death of the dopaminergic neurons in the pathological model animals with PD caused by different genetic abnormalities and even restore it to normal.

### Example 2: Effect of PD0325901

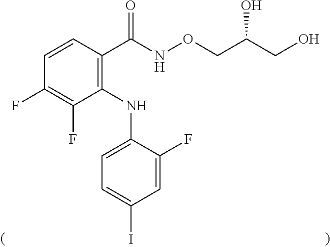 [SMILES: O=C(NOC[C@H](O)CO)c1ccc(F)c(F)c1Nc1ccc(I)cc1F]

### On Dopaminergic Neurons in Local Brain Regions

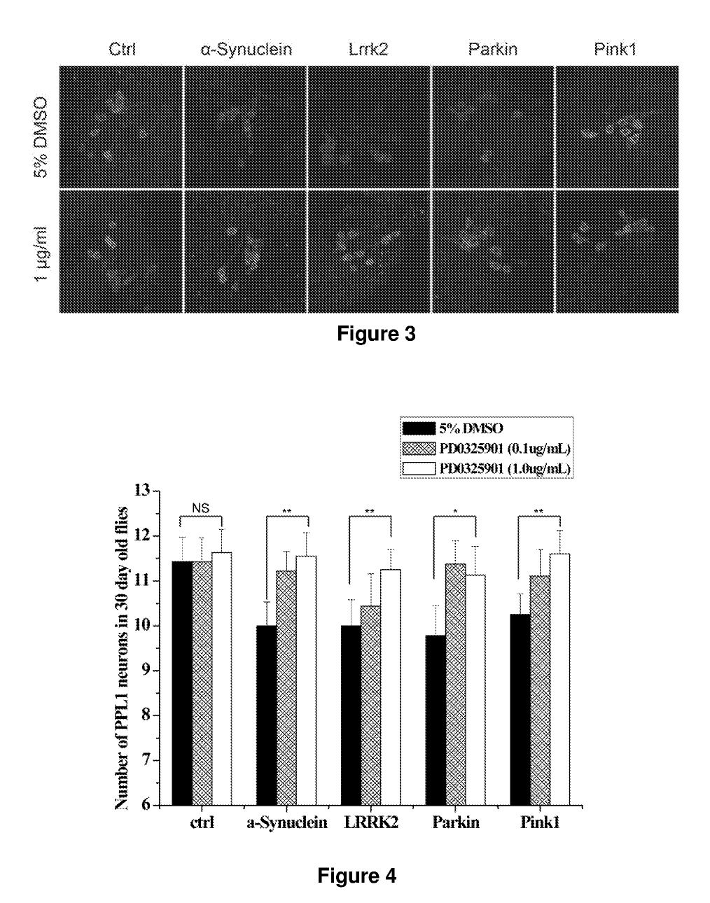

450 fruit flies of Drosophila melanogaster (purchased from Bloomington Drosophila Stock Center, USA) were divided into 4 PD disease model groups (as for “model groups”, each group has the same genotype and different groups have different genotypes; see the following) and 1 normal control group (Ctrl, non-PD type). Animal models of PD caused by alpha-synuclein transgene overexpression, Lrrk2 transgene overexpression, Parkin gene mutation and Pink1 gene mutation were established in the four model groups. Each group includes 90 fruit flies and each group was further equally divided into 2 drug-feeding groups and 1 non-drug-feeding control group for testing. Starting from imago at the age of 5 days, the drug-feeding groups were respectively continuously fed with 0.1 μg/mL, 1 μg/mL PD0325901 (PD0325901 is dissolved in 1% DMSO) for 30 days and the normal control group was fed with 5% DMSO as a solvent control. After 30 days of drug or control solvent feeding, the brains of fruit flies were dissected and the dopamine neurons were labeled by the whole-tissue immunostaining of the brains of the fruit flies with an anti-dopamine hydroxylase antibody. The morphology and quantity of the dopamine neurons in the PPL regions in the brains of the fruit flies were observed microscopically to determine the effect of PD0325901 on the dopaminergic neurons in the local brain regions of the fruit flies. The results thereof were shown in FIGS. 3 and 4.

FIG. 3 is a schematic diagram of the morpohology of dopaminergic neurons in the local brain region after treatment with PD0325901, wherein the fluorescent part is the cell body profile of the dopaminergic neurons, through which the morphology and quantity of the dopaminergic neurons can be observed. FIG. 4 is a statistical diagram of the quantity of the dopaminergic neurons in the PPL brain region after treatment with PD0325901. As can be seen from FIGS. 3 and 4, 1 μg/mL PD0325901 can effectively reverse the degenerative loss of the dopaminergic neurons in the pathological model animals with PD caused by different genetic abnormalities and even restore it to normal.

### Example 3: Effect of Trametinib (the Chemical Name is (N-(3-{3-cyanopropyl-5-[(2-fluoro-4-iodophenyl)amino]-6,8-dimethyl-2,4,7-trioxo-3,4,6,7-tetrahydropyrido[4,3-d]pyrimidin-1(2H)yl}phenyl)acetamide); the Molecular Formula is C₂₆H₂₃FIN₅O₄; the Structural Formula is

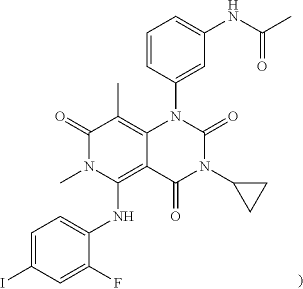 [SMILES: CC(=O)Nc1cccc(-n2c(=O)n(C3CC3)c(=O)c3c(Nc4ccc(I)cc4F)n(C)c(=O)c(C)c32)c1]

### On Dopaminergic Neurons in Local Brain Regions

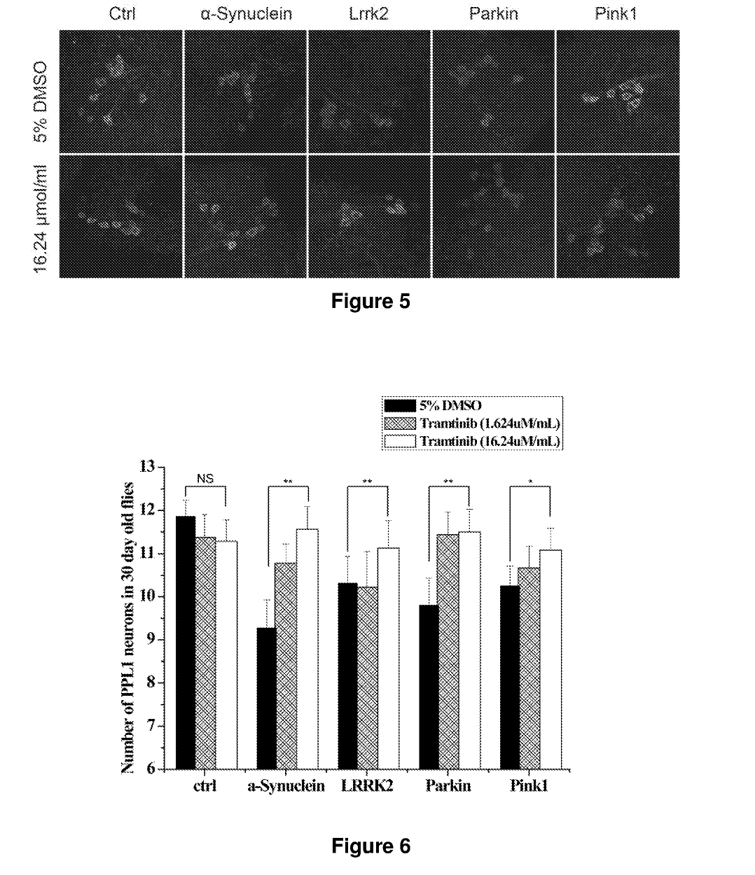

As experimental objects, 450 fruit flies of Drosophila melanogaster (purchased from Bloomington Drosophila Stock Center, USA) were divided into 4 PD disease model groups (as for “model groups”, each group has the same genotype and different groups have different genotypes; see the following) and 1 normal control group (Ctrl, non-PD type). Animal models of PD caused by alpha-synuclein transgene overexpression, Lrrk2 transgene overexpression, Parkin gene mutation and Pink1 gene mutation were established in the four model groups. Each group includes 90 fruit flies and each group was further equally divided into 2 drug-feeding groups and 1 non-drug-feeding control group for testing. Starting from imago at the age of 5 days, the drug-feeding groups were respectively continuously fed with 1.624 μM/mL, 16.24 μM/mL trametinib (trametinib is dissolved in 1% DMSO) for 30 days and the non-drug-feeding control group was fed with 5% DMSO as a solvent control. After 30 days of drug or control solvent feeding, the brains of fruit flies were dissected and the dopamine neurons were labeled by the whole-tissue immunostaining of the brains of the fruit flies with an anti-dopamine hydroxylase antibody. The morphology and quantity of the dopamine neurons in the PPL regions in the brains of the fruit flies were observed microscopically to determine the effect of trametinib on the dopaminergic neurons in the local brain regions of the fruit flies. The results thereof were shown in FIGS. 5 and 6.

FIG. 5 is a schematic diagram of the morphology of dopaminergic neurons in the local brain region after treatment with trametinib, wherein the fluorescent part is the cell body profile of the dopaminergic neurons, through which the state and quantity of the dopaminergic neurons can be observed. FIG. 6 is a statistical diagram of the quantity of the dopaminergic neurons in the PPL brain region after treatment with trametinib. As can be seen from FIGS. 5 and 6, 16.24 μM/mL trametinib can effectively reverse the degenerative death of the dopaminergic neurons in the pathological model animals with PD caused by different genetic abnormalities and even restore it to normal.

Therefore, by preparing MAPK signaling pathway inhibitors into medicaments for treating PD, the health of the dopaminergic neurons can be maintained and the patients with PD are expected to be treated fundamentally.

The above are only the preferred examples of the present invention and all equal changes and modifications made within the scope claimed in the present invention should belong to the scope of the present invention.

1. Use of an MAPK signaling pathway inhibitor in the manufacture of a medicament for delaying the degeneration of dopaminergic neurons.

2. Use of an MAPK signaling pathway inhibitor in the manufacture of a medicament for the treatment of Parkinson's Disease (PD).

3. The use according to claim 1, wherein the MAPK signaling pathway inhibitor is selected from the group consisting of an MEK inhibitor and an ERK antagonist.

4. The use according to claim 1, wherein the MAPK signaling pathway inhibitor is selected from the group consisting of U0126, PD0325901 and trametinib.

5. The use according to claim 1, wherein the MAPK signaling pathway inhibitor is trametinib.

6. A method for delaying the degeneration of dopaminergic neurons, comprising administering to a patient in need thereof a therapeutically effective amount of an MAPK signaling pathway inhibitor.

7. A method for treating Parkinson's Disease (PD), comprising administering to a patient in need thereof a therapeutically effective amount of an MAPK signaling pathway inhibitor.

8. The method according to claim 6, wherein the MAPK signaling pathway inhibitor is selected from the group consisting of an MEK inhibitor and an ERK antagonist.

9. The method according to claim 6, wherein the MAPK signaling pathway inhibitor is selected from the group consisting of U0126, PD0325901 and trametinib.

10. The method according to claim 6, wherein the MAPK signaling pathway inhibitor is trametinib.

11. An MAPK signaling pathway inhibitor for delaying the degeneration of dopaminergic neurons.

12. An MAPK signaling pathway inhibitor for the treatment of Parkinson's Disease (PD).

13. The signaling pathway inhibitor according to claim 11, which is selected from the group consisting of an MEK inhibitor and an ERK antagonist.

14. The signaling pathway inhibitor according to claim 11, which is selected from the group consisting of U0126, PD0325901 and trametinib.

15. The signaling pathway inhibitor according to claim 11, which is trametinib.

16. The use according to claim 2, wherein the MAPK signaling pathway inhibitor is selected from the group consisting of an MEK inhibitor and an ERK antagonist.

17. The use according to claim 2, wherein the MAPK signaling pathway inhibitor is selected from the group consisting of U0126, PD0325901 and trametinib.

18. The use according to claim 2, wherein the MAPK signaling pathway inhibitor is trametinib.

19. The method according to claim 7, wherein the MAPK signaling pathway inhibitor is selected from the group consisting of an MEK inhibitor and an ERK antagonist.

20. The method according to claim 7, wherein the MAPK signaling pathway inhibitor is selected from the group consisting of U0126, PD0325901 and trametinib.

21. The method according to claim 7, wherein the MAPK signaling pathway inhibitor is trametinib.

22. The signaling pathway inhibitor according to claim 12, which is selected from the group consisting of an MEK inhibitor and an ERK antagonist.

23. The signaling pathway inhibitor according to claim 12, which is selected from the group consisting of U0126, PD0325901 and trametinib.

24. The method according to claim 12, wherein the MAPK signaling pathway inhibitor is trametinib.
